(19) United States
(12) Patent Application Publication
(10) Pub. No.: US 2025/0170064 A1
(43) Pub. Date: May 29, 2025

(54) COMPOSITIONS COMPRISING AT LEAST ONE OF A TERPENE AND A CANNABINOID AND USES THEREOF

(71) Applicant: BUZZELET DEVELOPMENT AND TECHNOLOGIES LTD., Or Akiva (IL)

(72) Inventors: Aharon EYAL, Jerusalem (IL); Noa RAZ, Gizo (IL)

(21) Appl. No.: 18/843,326

(22) PCT Filed: Feb. 28, 2023
(86) PCT No.: PCT/IB2023/051839

§ 371 Date: Sep. 3, 2024

Related U.S. Application Data
(60) Provisional application No. 63/316,424, filed on Mar. 4, 2022.

(51) Int. Cl. A61K 9/14; A61K 9/00; A61K 31/00; A61K 31/015; A61K 31/045

(52) U.S. Cl. CPC A61K 9/143; A61K 9/007; A61K 31/015; A61K 31/045; A61K 31/658

(57) ABSTRACT

Provided is a composition comprising a carrier and at least one of a terpene and a cannabinoid, wherein the carrier comprises discrete particles at 30° C.; wherein the carrier comprises 50% wt to 99.9% wt of the total composition; wherein at least one of the terpene and the cannabinoid is carried on the carrier: wherein a total weight of the terpene and the cannabinoid forms 0.1% wt to 50% wt of the total composition: wherein the discrete particles each have a dimension in the range between 0.2 mm and 5 mm; and wherein at least 70% of the discrete particles each carry between 0.1 mg and 100 mg of the at least one of a terpene and a cannabinoid per particle.

### CROSS-REFERENCE TO RELATED APPLICATIONS

The present application gains priority from U.S. Provisional Patent Application Ser. No. 63/316,424 filed Mar. 4, 2022 which is incorporated by reference as if fully set-forth herein.

### FIELD OF THE INVENTION

The present invention relates to the field of compositions comprising terpenes and/or cannabinoids, and more specifically to such compositions comprising discrete particles, and uses thereof.

### BACKGROUND OF THE INVENTION

Enrichment of cannabis compositions with selected terpenes is known to be highly advantageous. Specific terpenes may be selected for use in treatment of specific conditions or symptoms thereof.

### SUMMARY OF THE INVENTION

According to an aspect of some embodiments of the present invention, there is provided a composition comprising a carrier and at least one of a terpene and a cannabinoid, wherein
(i) said carrier comprises discrete particles at 30° C.;
(ii) said carrier comprises 50% wt to 99% wt of said composition;
(iii) said at least one of a terpene and a carrier is carried on said carrier;
(iv) a total weight of said at least one of a terpene and a carrier forms 0.1% wt to 50% wt of said composition;
(v) said discrete particles each have a dimension in the range between 0.2 mm and 5 mm;
(vi) at least 70% of the discrete particles each carry between 0.1 mg and 100 mg of said at least one of a terpene and a cannabinoid per particle.

According to an aspect of some embodiments of the present invention, there is provided a kit comprising at least two compositions as disclosed herein, wherein a terpene content of a first of said compositions is different from a terpene content of a second of said compositions.

According to an aspect of some embodiments of the present invention, there is provided a method for enriching an air stream with at least one of a selected terpene and a selected cannabinoid, comprising contacting the air stream with the composition as disclosed herein, wherein a selected terpene and/or cannabinoid evaporates and combines with the air stream to form a terpene-enriched and/or a cannabinoid-enriched air stream.

According to an aspect of some embodiments of the present invention, there is provided a device for use in the method as disclosed herein, the device comprising an inlet, a contact area and an outlet, wherein said inlet is configured to allow entry of the air stream to said contact area, the contact area is configured to allow contact between the air stream and the composition to provide the terpene-enriched and or the cannabinoid-enriched air stream, and the outlet is configured for use in inhaling the enriched air stream.

According to an aspect of some embodiments of the present invention, there is provided a method for enriching a liquid with at least one of a selected terpene and a selected cannabinoid, comprising contacting said liquid with the composition as disclosed herein, wherein a terpene and/or a cannabinoid of said composition desorbs and combines with the liquid to form a terpene-enriched and/or a cannabinoid-enriched liquid.

### DETAILED DESCRIPTION OF THE INVENTION

The present invention relates to a composition comprising at least one of a terpene and a cannabinoid; and a carrier, wherein the composition is provided in the form of discrete particles.

The present invention provides terpene-enriched and/or cannabinoid-enriched compositions. Addition of terpenes to a cigarette comprising cannabis has been achieved by the present inventors using a device containing a terpene blend which, during use, is positioned between the cigarette and the mouth of the user, such that vapors comprising at least one cannabinoid are transferred through the device, thereby providing a terpene-enriched cannabinoid-containing vapor to the user. An inherent disadvantage of such use is that different terpenes evaporate at different rates, such that when used for smoking of multiple cigarettes, mainly the more volatile terpenes (such as monoterpenes) will be present in the vapor provided for the first number of cigarettes, while vapors provided for the later number of cigarettes will mainly include the less volatile terpenes. Use of a device to provide only the small amount of terpenes required for a single use (typically in the order of a few mg) is generally considered too expensive and therefore impractical. Other methods, such as dripping terpenes on the cigarette or filter are also considered impractical due to the need to control the amount of terpenes used.

The present inventors have addressed this problem by providing discrete particles (such as beads) onto which a selected amount of selected terpenes and/or selected cannabinoids is carried, wherein the particles are intended for single use, and wherein the user selects the required number of particles for each single use. The particles may be provided with various terpene and/or cannabinoid compositions and various terpene and/or cannabinoid amounts, and the user selects the required composition and required amount (either for therapeutic or recreational purposes).

Unless otherwise defined, all technical and scientific terms used herein have the same meaning as commonly understood by one of ordinary skill in the art to which this invention belongs.

The terminology used in the description of the invention herein is for describing particular embodiments only and is not intended to be limiting of the invention.

As used herein the term “cannabinoid-enriched” with regard to an air stream refers to an air stream in which the total cannabinoid concentration after contacting with the composition of the present invention is higher than prior to contacting with the composition. According to some embodiments, the total cannabinoid composition in the air stream is about 15%, 20%, 25%, 30%, 35%, 40%, 45%, 50%, 55%, 60%, 65%, 70%, 75%, 80%, 85%, 90% or 95% higher after contacting with the composition.

As used herein the term “terpene-enriched” with regard to an air stream refers to an air stream in which the total terpene concentration after contacting with the composition of the present invention is higher than prior to contacting with the composition. According to some embodiments, the total terpene composition in the air stream is about 15%, 20%, 25%, 30%, 35%, 40%, 45%, 50%, 55%, 60%, 65%, 70%, 75%, 80%, 85%, 90% or 95% higher after contacting with the composition.

As used in the description of the invention and the appended claims, the singular forms “a,” “an,” and “the” are intended to include the plural forms as well, unless the context clearly indicates otherwise.

As used herein, the term “treating” includes ameliorating, mitigating, and reducing the instances of a disease or condition, or the symptoms of a disease or condition.

As used herein, the term “therapeutically effective amount” means the amount of an active substance that, when administered to a subject for treating a disease, disorder, or other undesirable medical condition, is sufficient to have a beneficial effect with respect to that disease, disorder, or condition. The therapeutically effective amount will vary depending on the chemical identity and formulation form of the active substance, the disease or condition and its severity, and the age, weight, and other relevant characteristics of the patient to be treated. Determining the therapeutically effective amount of a given active substance is within the ordinary skill of the art and typically requires no more than routine experimentation.

Unless otherwise indicated, all numbers expressing quantities, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

As used herein, when a numerical value is preceded by the term “about”, the term “about” is intended to indicate +/−10% of that value.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”. As used herein, the terms “water content” and “moisture content” are used interchangeably.

Unless indicated otherwise, percent is weight percent and ratio is weight/weight ratio.

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

According to an aspect of some embodiments of the present invention, there is provided a composition comprising a carrier and at least one of a terpene and a cannabinoid, wherein
(i) the carrier comprises discrete particles at 30° C.;
(ii) the carrier comprises 50% wt to 99% wt of the composition;
(iii) the at least one of a terpene and a cannabinoid is carried (such as adsorbed or absorbed) on the carrier;
(iv) the total weight of the at least one of a terpene and a cannabinoid forms 0.1% wt to 50% wt of the composition;
(v) the discrete particles each have a dimension in the range between 0.2 mm and 5 mm;
(vi) at least 70% of the discrete particles each carry between 0.1 mg and 100 mg of the at least one of a terpene and a cannabinoid per particle.

According to some embodiments, at least 70%, at least 75%, at least 80%, at least 85%, at least 90%, at least 95% or even 100% of the discrete particles each carry between about 0.1 mg and about 100 mg of terpene per particle, such as about 0.1 mg, about 1 mg, about 10 mg, about 20 mg, about 30 mg, about 40 mg, about 50 mg, about 60 mg, about 70 mg, about 80 mg, about 90 mg, or about 100 mg, more preferably up to about 10 to 20 mg of terpene per particle.

According to some embodiments, at least 70%, at least 75%, at least 80%, at least 85%, at least 90%, at least 95% or even 100% of the discrete particles each carry between about 0.1 mg and about 100 mg of cannabinoid per particle, such as about 0.1 mg, about 1 mg, about 10 mg, about 20 mg, about 30 mg, about 40 mg, about 50 mg, about 60 mg, about 70 mg, about 80 mg, about 90 mg, or about 100 mg, more preferably up to about 10 to 20 mg of cannabinoid per particle.

According to some embodiments, the carrier comprises about 50% wt, about 60% wt, about 70% wt, about 80% wt, about 90% wt or up to 99% wt of the total composition. According to some embodiments, the carrier is a synthetic carrier. According to some embodiments, the carrier is of pharmaceutical grade. According to some embodiments, the carrier comprises silica. According to some embodiments, the carrier comprises silica beads. According to some embodiments, the carrier comprises granular active carbon. According to some embodiments, the carrier is porous. According to some embodiments, the carrier has a surface area of between 10 sqm (square meter) and 500 sqm per gram. According to some embodiments, the carrier surface is designed for adsorption of lipophilic matter.

According to some embodiments, the terpene comprises at least one monoterpene, selected from the group consisting of pinene, limonene, linalool, myrcene, terpineol, geraniol, borneol, and eucalyptol and combinations thereof.

According to some embodiments, the terpene comprises at least two monoterpenes, such as two, three, four, five, six, seven or more than seven monoterpenes.

According to some embodiments, the terpene further comprises at least one sesquiterpene, such as one, two, three, four, five or more than five sesquiterpenes.

According to some embodiments, the cannabinoid comprises at least one cannabinoid (such as one, two, three, four or five cannabinoids) selected from the group consisting of tetrahydrocannabinol (THC), cannabidiol (CBD), cannabigerol (CBG), cannabichromene (CBC) and cannabinol (CBN).

According to some embodiments, one or more of the cannabinoids is present in the composition in acid form. According to some embodiments, each of the cannabinoids is present in the composition in acid (carboxylated) form and/or in neutral (decarboxylated) form.

According to some embodiments, the particles each have a dimension of about 0.2 mm, about 0.5 mm, about 1 mm, about 1.5 mm or about 2 mm.

According to some embodiments, at least 90%, such as 90%, 92%, 95%, 98% or 100% of a total number of the particles is substantially spherical.

According to some embodiments, at least one of the terpene and the cannabinoid is provided in a synthetic form.

According to some embodiments, the composition is added to a cannabis cigarette to a tobacco cigarette or to a cigarette comprising both cannabis and tobacco by a user prior to smoking the cigarette. According to some embodiments, the composition is added to a cigarette comprising pre-extracted cannabis material. According to some embodiments, the composition is added to a filter of a cigarette.

According to an aspect of some embodiments of the present invention, there is provided a kit comprising at least two compositions as disclosed herein, wherein a terpene content of a first of the two compositions is different from a terpene content of a second of the two compositions. According to some embodiments, the first composition and the second composition comprise different terpenes. According to some embodiments, the first composition and the second composition comprise the same terpenes at different concentrations.

According to an aspect of some embodiments of the present invention, there is provided a kit comprising at least two compositions as disclosed herein, wherein a cannabinoid content of a first of the two compositions is different from a cannabinoid content of a second of the two compositions. According to some embodiments, the first composition and the second composition comprise different cannabinoids. According to some embodiments, the first composition and the second composition comprise the same cannabinoids at different concentrations.

According to an aspect of some embodiments of the present invention, there is provided a method for enriching an air stream with at least one of a selected terpene and a selected cannabinoid, comprising contacting the air stream with the composition as disclosed herein comprising a selected number of the discrete particles, wherein a selected terpene and/or cannabinoid of the composition evaporates and combines with the air stream to form a terpene-enriched and/or a cannabinoid-enriched air stream.

According to some embodiments, the air stream comprises a vapor of at least one cannabinoid prior to contacting with the composition. According to some embodiments, the at least one cannabinoid in the air stream prior to contacting is similar to the at least one cannabinoid of the composition with respect to the specific cannabinoid(s) present and/or the concentration therefore. According to some embodiments, the at least one cannabinoid in the air stream prior to contacting is different from the at least one cannabinoid of said composition.

According to some embodiments, the air stream comprises a vapor of at least one terpene prior to contacting with the composition. According to some embodiments, the at least one terpene in the air stream prior to contacting is similar to the at least one cannabinoid of the composition with respect to the specific terpene(s) present and/or the concentration therefore. According to some embodiments, the at least one terpene in the air stream prior to contacting is different from the at least one terpene of said composition.

According to some embodiments, each of the carrier (e.g. carrier surface area properties, such as hydrophilic/hydrophobic ratio), the terpene and/or the cannabinoid and/or the cannabinoid concentration and/or the terpene concentration are selected so that on contacting of the air stream with the composition, at least about 50%, such as about 50%, about 55%, about 60%, about 65%, about 70%, about 75%, about 80%, about 85%, about 90%, about 95%, or about 100% of the terpene content of the composition evaporates and/or so that at least 20% of the cannabinoid, such as about 20%, about 25%, about 30%, about 35%, about 40%, about 45%, about 50%, such as about 50%, about 55%, about 60%, about 65%, about 70%, about 75%, about 80%, about 85%, about 90%, about 95%, or about 100% of the terpene content of the composition evaporates.

According to some embodiments, the air stream is at a temperature higher than ambient temperature. According to some embodiments, the temperature of the air stream is adjusted to provide the desired extent of evaporation.

According to some embodiments, the moisture content of the air stream is different from that of ambient conditions. According to some embodiments, the moisture content of the air stream is adjusted to provide the desired extent of evaporation.

According to an aspect of some embodiments of the present invention, there is provided a device for use in the method as disclosed herein, the device comprising an inlet, a contact area and an outlet, wherein the inlet is configured to allow entry of air stream to the contact area, the contact area is configured to allow contact between the air stream and the composition to provide the terpene-enriched and/or the cannabinoid-enriched air stream, and the outlet is configured for use in inhaling the enriched air stream.

According to some embodiments, during use of the device, the composition is introduced into the contact area through the inlet. According to some embodiments, the device is provided for use with the composition within the contact area. According to some embodiments, the device is built for a single use. According to alternative embodiments, the device is built for multiple use.

According to some embodiments, the device is for use as a filter of a cigarette. According to some embodiments, the device is combined with a cannabis inhalation device, such as a vaporizer.

According to an aspect of some embodiments of the present invention, there is provided a method for enriching a liquid with at least one of a selected terpene and a selected cannabinoid, comprising contacting the liquid with the composition as disclosed herein comprising a selected number of the discrete particles, wherein a terpene and/or a cannabinoid of the composition desorbs and combines with the liquid to form a terpene-enriched and/or a cannabinoid-containing oil. According to some embodiments, the liquid comprises at least one cannabinoid prior to said contacting. According to some embodiments, the at least one cannabinoid in the liquid prior to contacting is similar to the at least one cannabinoid of the composition with respect to the specific cannabinoid(s) present and/or the concentration therefore. According to some embodiments, the at least one cannabinoid in the liquid prior to contacting is different from the at least one cannabinoid of said composition. According to some embodiments, the liquid comprises at least one terpene prior to said contacting. According to some embodiments, the at least one terpene in the liquid prior to contacting is similar to the at least one terpene of the composition with respect to the specific terpene(s) present and/or the concentration therefore. According to some embodiments, the at least one terpene in the liquid prior to contacting is different from the at least one terpene of said composition.

According to some embodiments, there is provided a method for providing a selected dose of a terpene-enriched and/or cannabinoid-enriched air stream to a subject, comprising administration of the enriched air stream.

According to some embodiments, there is provided a method for providing a terpene-enriched and/or cannabinoid-enriched vapor to a subject, comprising administration of the enriched air stream.

According to an aspect of some embodiments of the present invention, there is provided a method for providing a selected dose of a terpene and/or a cannabinoid to a subject, comprising administration of an enriched air stream and/or enriched liquid as disclosed herein. According to some embodiments, the subject is suffering from a condition to be treated by the enriched air stream and/or enriched liquid as disclosed herein. According to some embodiments, the subject is a recreational user of the composition as disclosed herein.

According to some embodiments, the selected number of particles is in the range of from 1 to 10, such as 1, 2, 3, 4, 5, 6, 7, 8, 9 or 10 particles.

According to some embodiments, there is provided a method for providing a selected dose of a terpene-enriched and/or a cannabinoid-enriched cannabinoid-containing oil to a subject, comprising administration of the terpene-enriched cannabinoid-containing oil as disclosed herein.

According to some embodiments, there is provided a method of treating a subject in a need thereof, comprising providing a selected dose of vapor as disclosed herein.

According to some embodiments, there is provided a method of treating a subject in a need thereof, comprising providing a selected dose of oil as disclosed herein.

According to some embodiments, the condition to be treated is selected from the group consisting of is selected from the group consisting of arthritis, osteoarthritis, arthralgia, joint pain, joint stiffness, diabetes, lack of appetite, anorexia, vomiting, nausea, an inflammatory bowel disease including Crohn's disease and ulcerative colitis, dementia, memory loss, osteoporosis, fatigue, weakness, decreased mental energy, decreased physical energy, dizziness, deficits in balance, itchy skin, pruritus and/or chronic pruritus, wrinkles, stretch marks, skin burn, headache, migraine, weight gain, digestive problem, intestinal disorder, gastrointestinal pain, abdominal pain, pelvic pain, constipation, diarrhea, hot flashes, sweating, difficulty in concentration, weakened immune system, cardiovascular disease, palpitation and tachycardia, psoriasis, dermatophytes, Candida, leishmaniasis, Methycillin-resistant Staphylococcus aureus (MRSA), malaria, allergy, fibromyalgia, nociceptive pain, neuropathic pain, pain, arm or leg pain, acne, insomnia and/or sleep disorders, muscle pain, myalgia, spasticity, muscle tension, cramps, spasms, anxiety, irritability, nervousness, restless, stress, depression, mood problem, affect disorders, anger, autism and/or autism spectrum disorder, neurodegenerative diseases, Alzheimer disease, Parkinson disease, Huntington's disease, Dystonia, Amyotrophic Lateral Sclerosis (ALS), multiple sclerosis, Tourette syndrome, Myasthenia Gravis, epilepsy, cancer, inflammatory airway diseases including asthma and/or chronic obstructive pulmonary disease (COPD), addiction, stroke, traumatic brain injury, motor spasm and/or tic, vocal tic, obsessive compulsive disorder (OCD), attention deficit hyperactivity disorder (ADHD), attention deficit disorder (ADD), cerebral palsy (CP), inflammation, oxidative stress, paresthesia, glaucoma and combinations thereof

### EXAMPLES

### Examples 1-11

| | | | Cannabinoid |
|---|---|---|---|
| | | Terpene | (Total |
| | Carrier | (Total terpene | cannabinoid |
| | (weight) | weight) | weight) |
| 1 | Silica (1 mg) | Linalool (0.2 mg) | |
| 2 | Silica (5 mg) | Limonene, Pinene, | |
| | | Geraniol, Borneol (1 mg) | |
| 3 | Silica (20 mg) | Myrcene, Terpineol, | |
| | | Borneol (10 mg) | |
| 4 | Silica (5 mg) | Pinene, Myrcene, | |
| | | Linalool, Caryophyllene, | |
| | | Humulene, Nerolidol, | |
| | | Bisabolol (1 mg) | |
| 5 | Silica (25 mg) | | THC (10 mg) |
| 6 | Silica (25 mg) | Pinene, Limonene, | |
| | | Terpinolene, | |
| | | Caryophyllene, Bisabolol | |
| | | (10 mg) | |
| 7 | Silica (15 mg) | Terpinolene (0.5 mg) | THC (5 mg) |
| 8 | Silica (25 mg) | Linalool, Myrcene, | THC, CBD |
| | | Caryophyllene (0.5 mg) | (10 mg) |
| 9 | Silica (25 mg) | Limonene (0.1 mg) | CBD (5 mg) |
| 10 | Silica (50 mg) | | CBD (20 mg) |
| 11 | Silica (2 mg) | Myrcene (0.5 mg) | |

### Examples 12-16

12. A pre-roll (a cigarette comprising cannabis plant material) composed of 1 gr cannabis inflorescence and 50 mg of silica particles, comprising 20 mg THC, embedded within the pre-roll's filter and top-end (to-be-lit end). Inhalation releases 20% of the THC.
13. A pre-roll composed of 0.5 gr cannabis inflorescence and 25 mg of silica particles, comprising 10 mg CBD, embedded within the pre-roll's filter and top-end (to-be-lit end). Inhalation releases 30% of the CBD.
14. A pre-roll composed of 0.5 gr cannabis inflorescence and 2 mg of silica particles, comprising 0.5 mg limonene, embedded within the pre-roll's filter and top-end (to-be-lit end). Inhalation releases 50% of the limonene.
15. 1 gr of ground cannabis inflorescence and 2 mg of silica particles, comprising 0.5 mg myrcene, to be used within a vaporizer. Inhalation releases 70% of the myrcene.
16. 1 gr of ground cannabis inflorescence and 20 mg of silica particles, comprising 10 mg CBD, to be used within a vaporizer. Inhalation releases 30% of the CBD.

1. A composition comprising a carrier and at least one of a terpene and a cannabinoid, wherein
(i) said carrier comprises discrete particles at 30° C.;
(ii) said carrier comprises 50% wt to 99.9% wt of said composition;
(iii) said at least one of a terpene and a cannabinoid is carried on said carrier;
(iv) a total weight of said at least one of a terpene and a cannabinoid forms 0.1% wt to 50% wt of said composition;
(v) said discrete particles each have at least one dimension in the range between 0.2 mm and 5 mm;
(vi) at least 70% of the discrete particles each carry between 0.1 mg and 100 mg of said at least one of a terpene and a cannabinoid per particle.

2. The composition of claim 1, wherein said terpene comprises at least one monoterpene, selected from the group consisting of pinene, limonene, linalool, myrcene, terpineol, geraniol, borneol, sabinene, terpinolene and eucalyptol and combinations thereof.

3. The composition of claim 1, wherein said terpene comprises at least two monoterpenes.

4. The composition of claim 1, wherein said terpene further comprises at least one sesquiterpene.

5. The composition of claim 1, wherein said cannabinoid comprises at least one cannabinoid selected from the group consisting of tetrahydrocannabinol (THC), cannabidiol (CBD), cannabigerol (CBG), cannabichromene (CBC) and cannabinol (CBN).

6. The composition of claim 1, wherein said at least one cannabinoid comprises at least two cannabinoids.

7. The composition of claim 1, wherein at least 60% of a total number of said particles is substantially spherical.

8. The composition of claim 1, wherein at least one of said terpene and said cannabinoid is provided in a synthetic form.

9. A kit comprising at least two compositions according to claim 1, wherein a terpene content of a first of said at least two compositions is different from a terpene content of a second of said at least two compositions.

10. A kit comprising at least two compositions according to claim 1, wherein a cannabinoid content of a first of said at least two compositions is different from a cannabinoid content of a second of said at least two compositions.

11. A method for enriching an air stream with at least one of a selected terpene and a selected cannabinoid, comprising contacting said air stream with the composition of claim 1 comprising a selected number of said discrete particles, wherein a selected terpene and/or cannabinoid of said composition evaporates and combines with the air stream to form a terpene-enriched and/or a cannabinoid enriched air stream.

12. The method of claim 11, wherein said air stream comprises a vapor of at least one cannabinoid and/or at least one terpene prior to contacting with said composition.

13. (canceled)

14. The method of claim 11, wherein each of the carrier, the terpene and/or the cannabinoid and/or the terpene concentration and/or the cannabinoid concentration are selected so that on contacting of said air stream with said composition, at least 50% of the terpene content and/or at least 20% of the cannabinoid content of said composition evaporates.

15. A device for use in the method of claim 11, the device comprising an inlet, a contact area and an outlet, wherein said inlet is configured to allow entry of the air stream to said contact area, the contact area is configured to allow contact between the air stream and the composition to provide the terpene-enriched and/or the cannabinoid-enriched air stream, and the outlet is configured for use in inhaling the enriched air stream.

16. A method for enriching a liquid with at least one of a selected terpene and a selected cannabinoid, comprising contacting said liquid with the composition of claim 1, wherein a terpene and/or a cannabinoid of said composition desorbs and combines with the liquid to form a terpene-enriched and/or a cannabinoid-enriched liquid.

17. The method of claim 16, wherein said liquid comprises cannabinoid.

18. (canceled)

19. The method of claim 11, wherein the selected number of particles is in the range of from 1 to 10.

20. A method for providing a selected dose of a terpene-enriched and/or cannabinoid-enriched air stream to a subject, comprising administration of the enriched air stream of claim 11.

21. A method for providing a selected dose of a terpene-enriched and/or cannabinoid-enriched vapor to a subject, comprising administration of the terpene enriched and/or cannabinoid-enriched-air stream of claim 12.

22. (canceled)

23. A method of treating a subject in a need thereof, comprising providing a selected dose of vapor according to claim 21.

24. (canceled)
